(19) United States
(12) Patent Application Publication
(10) Pub. No.: US 2008/0287894 A1
(43) Pub. Date: Nov. 20, 2008

(54) Urine Collecting Device

(76) Inventors: Eleanor Van Den Heuvel, Slough (GB); Felicity Jowitt, Middlesex (GB); Tara O'Connor, Worcestershire (GB)

Correspondence Address: Intellectual Property Dept.;Dewitt Ross & Stevens SC, 2 East Mifflin Street, Suite 600, Madison, WI, 53703-2865, US

(21) Appl. No.: 12/090,119

(22) PCT Filed: Oct. 13, 2006
(86) PCT No.: PCT/GB2006/003810

§ 371 Date: Jul. 17, 2008

(30) Foreign Application Priority Data
Oct. 13, 2005 (GB) 0520864.0

(51) Int. Cl. A61F 5/451

(52) U.S. Cl. 604/327

(57) ABSTRACT

A pad assembly for use with a urine collection device comprises an external layer (2), which is preferably an impermeable conformable material. The external layer (2) partially surrounds a filling material (3), which preferably comprises a single layer of open low-density material that does not appreciably resist the flow of urine nor retain urine. The pad assembly is preferably tapered and preferably is able to accommodate an end portion of a urine outlet tube (1) longitudinally within the boundary of the external layer (2).

The present invention relates to a pad for use with a urine collection device.

Millions of people world-wide are afflicted with the problem of urinary incontinence, the economic costs of this distressing condition are extremely high but the cost in terms of human suffering is incalculable. Both men and women are afflicted by the condition although many more women are affected and incidence increases with age.

For many younger men external catheter systems provide an adequate solution to the problem of severe urinary incontinence, but these systems are far less satisfactory for older men who are more likely to experience continence problems. For women continence management solutions are extremely limited. Internal catheters are associated with a host of medical problems, particularly urinary tract infections and urethral erosions. Large disposable pads to absorb urine are a safer option but are far from an ideal solution. Pads are bulky and require frequent changing if the wearer is to remain comfortable and free from odour problems. For immobile patients and those with sensory loss, wet pads can exacerbate the risk of pressure injury.

One of the major problems in designing a device to cope with incontinence is the very high flow rate which is a characteristic of urine flow. During a void the urine flow rate increases rapidly to a peak rate of 25-30 millilitres per second. To pump urine directly requires a powerful pump able to reach a high flow rate as soon as it is activated. This is extremely difficult to achieve and would require a much larger pump and power source than can be easily transported.

WO 00/57784, the entire contents of which are incorporated herein by reference, discloses a urine collection device in which a vacuum is maintained. When urine is detected at an interface between the device and its user, a valve is opened allowing the pumping of urine into a storage container. In this device, a layered interface between the device and its user is provided. The interface preferably comprises an outer layer that is impervious to air and liquids, above which is a spacing layer through which liquid can flow. A nylon mesh layer is provided through which the urine must flow before entering the urine outlet tube. A layer of shape-retaining material is provided to give structural stability to the interface, and to allow urine to flow along the length of the interface into a reservoir section. Finally, an upper receiving layer takes up urine and allows it to flow through to the lower layers.

The present invention seeks to provide an improved interface that may be used with the device disclosed in WO 00/57784. In particular, the improved interface seeks to reduce leakage that could result from the use of several different layers, which can slow the passage of urine through the interface towards the urine outlet tube.

According to a first aspect of the present invention there is provided a pad assembly for use at a non-invasive interface of the urine collection device and its user, the pad assembly able to receive urine and channel it to a urine outlet tube; comprising: a liquid-impermeable exterior, the exterior including a urine-receiving hole; and a layer of flexible filling material through which urine is able to flow substantially freely, the filling material being partially surrounded by the exterior.

A pad assembly having these features allows urine to flow through without substantial build up within the pad assembly. It enables collection of urine from a user for delivery to a urine collection device whilst reducing problems of leakage.

Preferably the filling material is not multi-layer. In the preferred embodiment the filling material comprises a single layer. Provision of several different layers could slow the passage of urine through the interface and may result in leakage.

The filling material preferably allows urine to flow through at a rate of at least 25 ml per second, more preferably at a rate of 30 ml per second, even more preferably at a rate of at least 35 ml per second. Urine flow rate can increase to a peak of 25-30 ml per second. Such filling material therefore allows urine to flow through without substantially restricting its flow.

In the preferred embodiment, the filling material comprises an open, low density material. Such material is suitable and allows urine to flow through substantially freely.

Preferably, the filling material has a density of 0.03-0.01 g cm³. This is suitable for a substantially free flow of urine.

The porosity of the filling material is preferably between 50% and 99% of its volume, more preferably between 60% and 99%, more preferably between 70% and 99%, more preferably between 75% and 99%, more preferably between 80% and 99%, more preferably between 85% and 99%, more preferably between 90% and 99%, and even more preferably between 95% and 99%. These porosities facilitate an unrestricted flow of urine.

The filling material may comprise a reticulated foam, a non-woven plastic fibre or a woven spacer fabric. These have been identified as suitable materials.

In the preferred embodiment, the interior of the pad assembly is able to accommodate an end portion of an urine outlet tube such that, in use, at least a portion of a urine outlet tube is accommodated longitudinally within the boundary of the exterior. This provides for a more compact arrangement, which helps maintain the urine outlet tube in position. It also serves to reduce leakage problems.

One end of the pad may be narrower than its opposite end. This facilitates channeling of urine to the urine outlet tube.

According to a second aspect of the present invention there is provided a pad assembly for use at a non-invasive interface of a urine collection device and its user, the pad assembly able to receive urine and channel it to a urine outlet tube; comprising: a liquid-impermeable exterior, the exterior including a urine-receiving hole; and a layer of flexible filling material through which urine is able to flow, the filling material being partially surrounded by the exterior; wherein the interior of the pad assembly is able to accommodate an end portion of a urine outlet tube such that, in use, at least a portion of a urine outlet tube is accommodated longitudinally within the boundary of the exterior.

This arrangement helps keep the urine outlet tube in place and helps to prevent leakage between the pad assembly and the urine outlet tube.

The urine outlet tube could be accommodated within the filling material, or between the filling material and the exterior. Whilst the second arrangement may be simpler, the first arrangement can result in a tube that is not visible along the lower part of the pad assembly.

In an embodiment, the pad assembly is able to accommodate the urine outlet tube along its full length. This enables the inlet to the outlet tube to be positioned at the lowest part (in use) of the pad assembly, which aids in urine collection.

In the preferred embodiment, urine is able to flow substantially freely through the filling material. This allows urine to flow through without substantial build up within the pad assembly. It enables collection of urine from a user for delivery to a urine collection device whilst reducing problems of leakage.

Preferably a first end of the pad assembly is narrower than its opposite end. This facilitates channeling of urine to the urine outlet tube.

According to a third aspect of the present invention there is provided a pad assembly for use at a non-invasive interface of a urine collection device and its user, the pad able to receive urine and channel it to a urine outlet tube; comprising: a liquid-impermeable exterior, the exterior including a urine-receiving hole; and a layer of flexible filling material through which urine is able to flow, the filling material being partially surrounded by the exterior; wherein a first end of the pad assembly is narrower than its opposite end.

A pad assembly having this configuration facilitates channeling of the urine to the urine outlet tube. This helps to reduce leakage problems.

The pad assembly preferably includes a taper at its first narrow end. This further facilitates urine channeling to the outlet tube.

The first narrow end of the pad assembly is preferably blunt and is preferably substantially the same size as the diameter of a urine outlet tube. This provides a suitable location for a urine outlet tube and helps to prevent urine leakage.

Preferably, a second end of the pad is rounded. This provides comfort for the user.

In the preferred embodiment, urine is able to flow substantially freely through the filling material. This allows urine to flow through without substantial build up within the pad assembly. It enables collection of urine from a user for delivery to a urine collection device whilst reducing problems of leakage.

Preferably, the interior of the pad is able to accommodate an end portion of a urine outlet tube such that, in use, at least a portion of a urine outlet tube is accommodated longitudinally within the boundary of the exterior. This arrangement helps keep the urine outlet tube in place and helps to prevent leakage between the pad assembly and the urine outlet tube.

Preferably the exterior of the pad assembly is conformable. This makes the pad more comfortable for the user.

The pad assembly preferably has a capacity of less than 200 ml. A small pad assembly is less intrusive for the user.

According to a fourth aspect of the present invention there is provided a urine collection device including a pad assembly.

Preferred embodiments of the present invention are described below, by way of example only, and with reference to the accompanying drawings, in which:

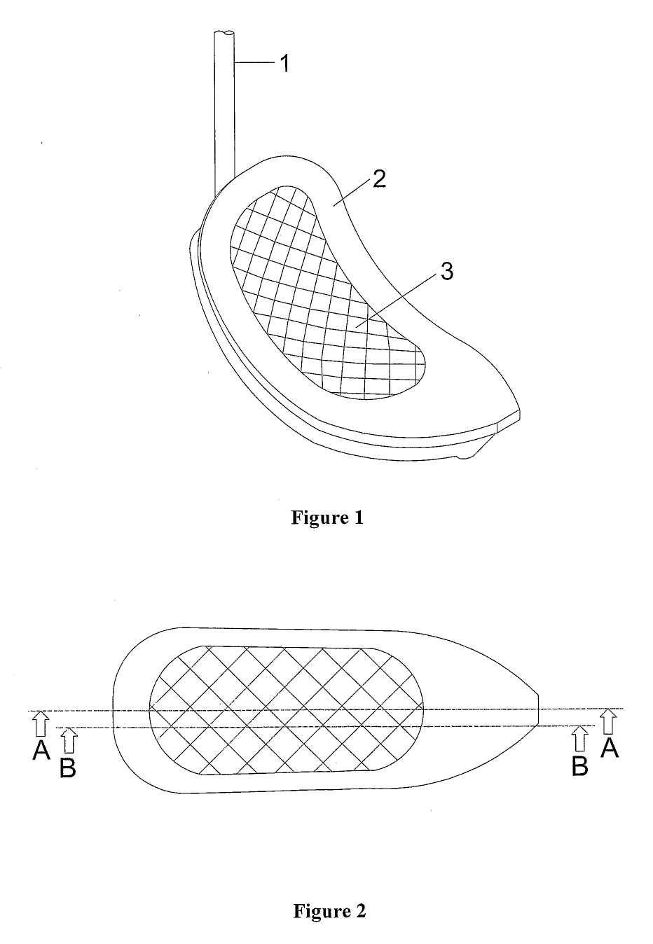

FIG. 1 shows an isometric view of a pad according to an embodiment of the invention;

FIG. 2 shows a top view of the pad of FIG. 1;

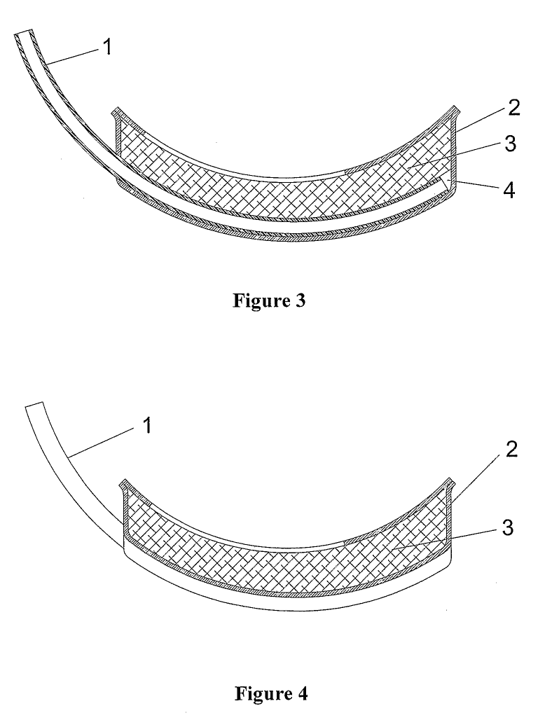

FIG. 3 shows a longitudinal cross section along the line A-A of FIG. 2; and

FIG. 4 shows a longitudinal cross section along line B-B on FIG. 2.

Referring to FIGS. 1 and 2, a preferred pad comprises an external layer 2 that partially surrounds filling material 3. The filling material 3 comprises a single layer of open low-density material that does not appreciably resist the flow of urine nor retain urine, and thereby allows urine to pass rapidly though. An example of a suitable material would be one that is non-woven, made up of fibres with open spaces between, such as the material used in scouring pads. The fibres may be tangled or may be held together with glue or resin. A suitable filling material may be described as a non-woven material made from tangled polymeric fibre to give a disorganised open web structure. The filling material 3 should be flexible and comfortable to sit on whilst retaining the open space within it.

The filling material 3 resists crushing but is still comfortable to wear. The density of the filling material 3 in this embodiment is 0.03-0.1 g/cm³. The porosity of the filling material 3 is in this embodiment between 50% and 99% of the volume of the pad. Water should be able to enter the material at a flow rate of at least 35 ml per second without surface run off and be able to run freely though the material towards a urine outlet tube.

The external layer 2 is an impermeable conformable material such as flexible plastic or expanded polystyrene. The external layer 2 should be reasonably soft, but able to retain its shape. It covers the filling material 3 apart from a rounded, rectangular hole in a position to correspond with the urethral opening of a user.

As shown in FIGS. 1 and 2, the pad has an upper rounded end (to be worn towards the front by a user), and a lower tapered end (to be worn towards the back by a user). This shape provides a more comfortable fit for the user, and helps to prevent leakage. It also aids channeling of urine to the urine outlet tube.

FIGS. 3 and 4 illustrate the arrangement of the filling material 3 within the external layer 2. The urine outlet tube 1 enters the pad at its upper (front) end and extends longitudinally along the full length of the pad, underneath the filling material 3, but inside the external layer 2. The open end of the urine outlet tube 1 fits within the tapered end 4 of the pad. In this embodiment, the urine outlet tube 1 has been accommodated by shaping the external impervious layer 2 around the outside of the tube.

The user places the pad inside their underwear. Specially adapted underwear including a mesh overlying the gusset could be used. The mesh serves to hold the pad in place.

Urine is received by the filling material 3 exposed through the hole in the external layer 2. Since the urine is able to flow freely through the filling material 3, gravity draws it towards the tapered end 4 of the pad and leakage is avoided. The end of the urine outlet tube 1 is located at the tapered end 4 of the pad. Urine is thus channeled towards the tapered end 4 of the pad from where it can be removed by the urine outlet tube 1.

There are several advantages of the above-described arrangement.

The single layer of filling material 3 ensures a rapid flow of urine through the pad and to the outlet tube, thereby minimising the possibility of leakage.

The location of the urine outlet tube 1 inside the external layer 2 ensures a good connection between the urine outlet tube 1 and the pad, which also helps to reduce the likelihood of leakage. The need for a specific connector between the urine outlet tube 1 and the pad is avoided. This arrangement is cheaper to produce than that previously described.

The rounded, upper end of the pad provides a comfortable fit, whilst the tapered, lower end 4 facilitates channeling of the urine to the urine outlet tube 1.

Various modifications can be made to the above-described arrangement.

The filling material 3 can be any suitable material that gives no effective resistance to a flow of urine. In other words, the urine should pass through the filling material as though it were not present. In particular, suitable materials for the filling material 3 include a reticulated foam, a non-woven plastic fibre, a woven spacer fabric or any similar low-density material.

The external layer 2 can be made from any suitable material having the desired properties. Suitable materials are described in WO 00/57784. Other suitable materials for the external layer 2 include impermeable materials such as a thin, soft closed cell foam or a closed cell polyethylene substance such as Plastazote® (a closed cell cross-linked polyethylene foam).

The hole in the external layer 2 through which urine is able to pass need not be rectangular. It could, for example, be oval, or any other suitable shape.

If the filling material 3 is a substance able to hold its shape well, the external layer 2 need not be anything more substantial than a simple impermeable cover.

Other arrangements for the urine outlet tube 1 within the pad can be envisaged. For example, the tube could be accommodated within the filling material 3 so that it is not visible along the lower part of the pad.

The skilled person will appreciate that the various modifications can be substituted or combined as appropriate.

The disclosures in British Patent Application No. GB 0520864.0 from which this application claims priority, and in the abstract accompanying this application are incorporated herein by reference.

1-27. (canceled)

28. A pad assembly for use as a non-invasive interface of a urine collection device, comprising:
a. a liquid-impermeable external layer including a urine-receiving aperture, an upper end and a lower end;
b. a layer of flexible filling material through which urine is able to flow substantially freely, the filling material being partially surrounded by the external layer; and
c. an outlet tube for use in the withdrawal of urine from the pad assembly, the outlet tube extending through the external layer to the lower end of the pad assembly.

29. The pad assembly of claim 28 wherein the filling material comprises a single layer.

30. The pad assembly of claim 28 wherein the filling material allows urine to flow through at a rate of at least 25 ml/second.

31. The pad assembly of claim 30 wherein the filling material allows urine to flow through at a rate of at least 35 ml/second.

32. The pad assembly of claim 28 wherein the filling material comprises an open, low-density material.

33. The pad assembly of claim 32 wherein the filling material has a density of 0.03-0.1 g/cm3.

34. The pad assembly of claim 32 wherein the porosity of the filling material is between 50% and 99% of its volume.

35. The pad assembly of claim 32 wherein the filling material includes one or more of reticulated foam, a non-woven plastic fibre or a woven spacer fabric.

36. The pad assembly of claim 28 wherein the urine outlet tube extends within the filling material.

37. The pad assembly of claim 28 wherein at least a portion of the urine outlet tube is accommodated longitudinally within the boundary of the external layer.

38. The pad assembly of claim 28 wherein the lower end of the pad assembly is narrower than its upper end.

39. The pad assembly of claim 28 wherein the lower end of the pad assembly is blunt and substantially the same size as the diameter of the outlet tube.

40. The pad assembly of claim 28 wherein the outlet tube extends along the full length of the pad.

41. The pad assembly of claim 28 wherein the exterior is conformable.

42. A urine collection device including a pad assembly comprising:
a. a liquid-impermeable external layer including:
(1) an outer face, and
(2) a collection face opposite the outer face, the collection face being bounded by a collection face edge and including:
(a) a urine-receiving aperture spaced inwardly from the collection face edge,
(b) a length extending between opposing upper and lower ends situated along the collection face edge, with the collection face narrowing as it approaches the lower end;
wherein a pad interior is defined between the outer face and the collection face, the pad interior opening onto the urine-receiving aperture;
b. a layer of flexible filling material through which urine is able to flow substantially freely, the filling material being situated within the pad interior; and
c. an outlet tube for use in the withdrawal of urine from the pad assembly, the outlet tube extending from the lower end of the pad assembly and opening adjacent the filling material, whereby urine flowing through the filling material may enter the outlet tube.

43. The urine collection device of claim 42 wherein the urine-receiving aperture is spaced further from the lower end than the upper end.

44. The urine collection device of claim 42 wherein the outlet tube extends:
a. through the external layer at or near the upper end,
b. within the pad interior to a location at or near the lower end.

45. The urine collection device of claim 42 wherein the outlet tube extends upwardly away from the upper end.

46. The urine collection device of claim 42 wherein the filling material is situated between the urine-receiving aperture and the outlet tube.

47. A urine collection device including a pad assembly comprising:
a. a liquid-impermeable external layer including:
(1) a convex outer face, and
(2) a continuous collection face opposite the outer face, the collection face having:
(a) a length extending between opposing upper and lower collection face ends situated along a collection face edge, and
(b) a urine-receiving aperture spaced inwardly from the collection face edge, and being spaced closer to the upper collection face end than to the lower collection face end,
wherein a pad interior is defined between the outer face and the collection face, the pad interior opening onto the urine-receiving aperture;
b. an outlet tube for use in the withdrawal of urine from the pad assembly, the outlet tube extending from the lower end of the pad assembly and opening onto the pad interior;
c. a layer of flexible filling material through which urine is able to flow substantially freely, the filling material being situated within the pad interior between the outlet tube and the urine-receiving aperture.
